(19) United States
(12) Patent Application Publication
(10) Pub. No.: US 2021/0113761 A1
(43) Pub. Date: Apr. 22, 2021

(54) IRRIGATION SYSTEM WITH PRESSURE SENSING AND CONTROL

(71) Applicant: Coloplast A/S, Humlebaek (DK)

(72) Inventors: Niels HVID, Vedbaek (DK); Henrik BAY, Lyngby (DK)

(21) Appl. No.: 16/498,396

(22) PCT Filed: Apr. 10, 2018
(86) PCT No.: PCT/DK2018/050073

§ 371 Date: Sep. 27, 2019

(30) Foreign Application Priority Data
Oct. 4, 2017 (DK) PA 2017 70260

(51) Int. Cl. A61M 3/02; A61M 39/10

(52) U.S. Cl. CPC A61M 3/0216; A61M 39/10; A61M 2210/1067; A61M 2205/3344; A61M 2205/3334; A61M 3/0258

(57) ABSTRACT

An irrigation system for anal or stomal irrigation includes a reservoir, a tubing system providing a first flow path between the reservoir and a catheter, and a pressure channel separate from the first flow path. A pump is operable to move the liquid through the first flow path from the reservoir and through the catheter. A control system is connected to the tubing system. A connector couples the tubing system to the catheter, where the connector includes a chamber coupling the pressure channel to the first flow path, with a membrane located in the chamber between the first flow path and the pressure channel. A pressure sensor communicates with the control system which operates the pump to control the anal or the stomal irrigation with the liquid based on the pressure sensed in the first flow path.

The present disclosure relates to an irrigation system for anal and/or stomal irrigation and related method. In particular, a method of operating an irrigation system for anal and/or stomal irrigation is disclosed.

### BACKGROUND

Control of voluntary bowel functions is frequently limited or absent in patients suffering from certain disabilities, such as spinal injuries, multiple sclerosis or spina bifida. Such lack of control of voluntary bowel functions typically results in faecal incontinence or intractable constipation, as patients have significantly reduced ability to sense the presence of faeces in the colon terminal part and the rectum and to sense the evacuation stimulus. Patients having undergone stomal surgery wherein a catheterizable stoma is constructed may suffer from similar difficulties.

It is known to accomplish bowel emptying by irrigation (i.e. flushing) of the rectum or stoma, by an irrigating fluid, such as tap water or saline, which is provided through an intermittent catheter with a tip which is configured and sized for insertion into the rectum or stoma, where it remains in a fixed position by an expandable inflation element, such as a balloon. The balloon may be inflatable by air or by water. Once the rectum or stoma has been flushed with the irrigation liquid, the expandable retention element is allowed to collapse to its non-deflated state, allowing the catheter to be withdrawn from the rectum or stoma, and allowing irrigation liquid and faeces to evacuate. The catheter is connected to a reservoir of irrigation liquid through a tube, and a pump may be provided for displacing or pumping irrigation liquid from the reservoir to the catheter.

### SUMMARY

There is a need for systems and/or methods increasing the safety for a user of an irrigation system and/or reduce the risk of damage or irritation of a user's bowel during and/or after irrigation.

Accordingly, an irrigation system for anal and/or stomal irrigation is provided, the irrigation system comprising a reservoir for an irrigation liquid; a tubing system with a first end connectable to the reservoir, and a second end connectable to a catheter for provision of a first fluid path for the irrigation liquid between the reservoir and the catheter comprising a distal end for expelling irrigation liquid from the catheter; one or more pressure sensors including a first pressure sensor for provision of a first pressure signal; a pump operable to pump the irrigation liquid from the reservoir to the second end of the tubing system; and a control system connected to the first pressure sensor and configured to operate the pump based on the first pressure signal. The tubing system optionally comprises a pressure channel and a first membrane. The first membrane can be arranged between the first fluid path and the pressure channel, and optionally the first pressure sensor is configured to measure the pressure in the pressure channel.

Also, a method of operating an irrigation system for anal and/or stomal irrigation is provided. The irrigation system optionally comprises a reservoir for an irrigation liquid; a tubing system with a first end connectable to the reservoir, and a second end connectable to a catheter for provision of a first fluid path for the irrigation liquid between the reservoir and the catheter comprising a distal end for expelling irrigation liquid from the catheter; one or more pressure sensors including a first pressure sensor for provision of a first pressure signal; a pump operable to pump the irrigation liquid from the reservoir to the second end of the tubing system; and a control system connected to the first pressure sensor. and configured to operate the pump based on the first pressure signal, the tubing system comprising a pressure channel and a first membrane, wherein the first membrane is arranged between the first fluid path and the pressure channel, and the first pressure sensor is configured to measure the pressure in the pressure channel. The method comprises: pumping irrigation liquid from the reservoir through the first fluid path; measuring the pressure in the pressure channel, and reducing the irrigation liquid pressure in the first fluid path if the pressure in the pressure channel is larger than a first pressure threshold.

The present disclosure enables more safe anal and/or stomal irrigation by improving the pressure control of the irrigation liquid flow in the irrigation system. The present disclosure enables more effective irrigation while at the same time providing a safe irrigation, by improving the accuracy of fluid pressure control in the irrigation system. Current systems, in order to comply with safety regulations, typically operate with a large safety margin, which in turn reduces the irrigation efficiency in certain use situations. The safety margins applied may result in irrigation liquid pressure at the catheter tip below a desired minimum pressure, e.g. in the range from 1.2 to 1.6 psi, for certain use situations. It is an advantage of the present disclosure that the liquid pressure control is improved, e.g. by increasing the minimum liquid pressure of the irrigation liquid at the catheter tip.

The present disclosure provides a simple, efficient, and easy-to-use irrigation system with a high degree of safety for a user, in particular for a POC system that can be used in many setups/situations.

### BRIEF DESCRIPTION OF THE DRAWINGS

The accompanying drawings are included to provide a further understanding of embodiments and are incorporated into and a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 illustrates an exemplary irrigation system,

FIG. 2 illustrates an exemplary irrigation system,

FIG. 3 illustrates a part of an exemplary tubing system with a catheter,

FIG. 4 schematically illustrates a part of an exemplary irrigation system,

FIG. 5 schematically illustrates a part of an exemplary irrigation system, and

FIG. 6 shows a more detailed view of a part of the second tubing part.

### DETAILED DESCRIPTION

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

An irrigation system for anal and/or stomal irrigation is disclosed. The irrigation system may be a point-of-care (POC) irrigation system. A POC irrigation system must be safe to operate with low or no risk of errors, in particular with no risk of self-damaging of a user. The irrigation system may be an irrigation system with no return flow of fluid and faecal material in the tubing system of the irrigation system.

The irrigation system comprises a reservoir for an irrigation liquid. The reservoir may be arranged in a reservoir housing. The reservoir may be configured to hold irrigation liquid having a volume of in the range from 100 mL to 3.0 L. In one or more exemplary irrigation systems, the reservoir has a reservoir volume less than 3 L.

The irrigation system optionally comprises a user interface, optionally arranged in a user interface housing and/or the reservoir housing. The user interface is configured to control operation of the irrigation system, e.g. by sending “start”, “stop”, and/or “pause” control signals to the control system in response to a user providing user input, e.g. by pressing one or more buttons of the user interface. The user interface may comprise a touch sensitive surface, such as a touch-screen for receiving user input.

The irrigation system comprises a tubing system with a first end and a second end. The first end is optionally connectable or connected to the reservoir and the second end is optionally connectable or connected to a catheter or an adapter for provision of a first fluid path for the irrigation liquid between the reservoir and the catheter comprising a distal end for expelling irrigation liquid from the catheter. The irrigation system may comprise a first fluid path from the first end of the tubing system to the second end of the tubing system. The irrigation system may comprise a second fluid path. The second fluid path may extend to the second end of the tubing system. The second fluid path may extend from the first end of the tubing system to the second end of the tubing system. The second fluid path may be parallel to the first fluid path for feeding second fluid to the second end of the tubing system.

The tubing system may comprise one or more tubing parts, such as a first tubing part and/or a second tubing part, for forming fluid path(s) between different housings of the irrigation system. The tubing system and parts thereof may comprise one or more first channels for forming parts of the first fluid path. The tubing system and parts thereof may comprise one or more second channels for forming parts of a second fluid path. The first fluid path of the irrigation system is for feeding irrigation liquid from the reservoir to the second end of the tubing system, e.g. for irrigating the bowel of the user. The second fluid path of the irrigation system may be for feeding irrigation liquid from the reservoir to the second end of the tubing system. The second fluid path may be for inflating or filling a balloon of the catheter, e.g. with irrigation liquid from the reservoir and/or air.

The tubing system may comprise a first connector at the first end of the tubing system. The tubing system may comprise a second connector at the second end of the tubing system. A tubing system with one or more connectors facilitate the use of different sized tubing system, e.g. to adapt to different users, and/or easy handling of the irrigation system, e.g. disassembly for storing. The second connector may be configured for coupling, e.g. detachable coupling, to a catheter and/or a catheter adapter.

The irrigation system comprises one or more pressure sensors including a first pressure sensor for provision of a first pressure signal. The one or more pressure sensor may comprise a plurality of pressure sensors. The one or more pressure sensors may include a second pressure sensor for provision of a second pressure signal. The control system may be connected to the second pressure sensor. The control system may be configured to operate the pump based on the second pressure signal. A second pressure sensor increases the robustness of the pressure control and/or reduces the risk of errors in the irrigation system. The second pressure sensor may be arranged in a user interface housing or in the reservoir housing. The one or more pressure sensors may include a third pressure sensor for provision of a third pressure signal. The control system may be connected to the third pressure sensor. The control system may be configured to operate the pump based on the third pressure signal. The third pressure sensor may be arranged in the user interface housing or in the reservoir housing.

The first pressure sensor provides a first pressure signal. The first pressure signal may be indicative of relative pressure between the pressure in the pressure channel and an ambient pressure. The first pressure signal may be indicative of absolute pressure in the pressure channel. The first pressure sensor may be arranged in the user interface housing or in the reservoir housing.

The irrigation system comprises a pump, such as an electrical pump, operable to pump the irrigation liquid from the reservoir to the second end of the tubing system. The pump may be arranged with the reservoir in the reservoir housing. Arranging the pump in the reservoir housing allows for a small user interface housing. Arranging the pump in the reservoir housing allows for a more precise control of the irrigation flow in the first fluid path. The pump may be arranged in the user interface housing. Arranging the pump in the user interface housing allows for a more simple first tubing part with no wired connection.

The irrigation system comprises a control system comprising a controller connected to the first pressure sensor. The control system may be configured to operate the pump based on the first pressure signal. The control system may be configured to operate the pump based on the second pressure signal. The control system may be configured to operate the pump to reduce the irrigation liquid pressure in the first fluid path, if the pressure in the pressure channel is larger than a first pressure threshold and/or fulfils a pressure criterion.

The tubing system comprises a pressure channel and a first membrane, wherein the first membrane is arranged between the first fluid path and the pressure channel. The first membrane is flexible such that the liquid pressure in the first fluid path is transferred, e.g. at least within a first pressure range, to the pressure channel. The tubing system may comprise a second connector at the second end, and the first membrane may be arranged in the second connector. The first membrane may also be denoted a diaphragm and operates to eliminate, minimize or reduce the pressure difference between the pressure in the first fluid path and the pressure in the pressure channel. Thereby, the liquid pressure in the first fluid path at the position of the first membrane can be translated to a pressure sensor at a distance from the first membrane via the pressure channel. The pressure channel may be filled with air, thereby avoiding the pressure contribution from a liquid in the pressure channel.

The second connector comprises one or more connector parts including a first connector part, optionally a second connector part, optionally a third connector part 46. The first connector part may, in an assembled second connector, be optionally snap-locked to and/or in threaded engagement with the second connector part. The second connector part may, in an assembled second connector, be optionally snap-locked to and/or in threaded engagement with the third connector part. The second connector has a first outlet of first fluid path and optionally a second outlet of the second fluid path (if present) of the tubing system 6. The first membrane may be arranged in an expansion chamber of the first connector part. Thereby, the surface area of the flexible first membrane is sufficiently large to reduce, minimize or eliminate the pressure difference between the first fluid path and the pressure channel. The first membrane separates the first fluid channel and the pressure channel. The first membrane is flexible to substantially equalize the pressure in the first fluid path and the pressure in the pressure channel, e.g. at least within a pressure difference less than a pressure difference threshold. The pressure difference threshold may be in the range from 0 to 0.5 psi, such as less than 0.2 psi. The second connector may have a first channel forming a part of the first fluid path, the first channel ending in the first outlet. The second connector may have a second channel forming a part of the second fluid path, the second channel ending in the second outlet.

The second connector may comprise a first window between a first channel in the second connector and the pressure channel, the first channel in the second connector optionally forming part of the first fluid path. The first window may be sealed by the first membrane. The first membrane may be arranged at a distance less than 50 cm from the second end of the tubing system. The first membrane may have a first surface with a first surface area, the first surface facing the first fluid path. The first membrane may have a second surface with a second surface area, the second surface facing the pressure channel. The first membrane may have a first surface area less than 5 cm², such as in the range from 10 mm² to 4 cm². The first membrane may have a thickness less than 1 mm.

The first pressure sensor is optionally configured to measure the pressure in the pressure channel. The second pressure sensor is optionally configured to measure the pressure in the pressure channel. The first pressure sensor may be arranged in the reservoir housing accommodating the reservoir.

The irrigation system may comprise a user control interface housing with a user control interface. The user control interface enables a user to control the irrigation system. The user control interface housing may be attached to the tubing system. The first pressure sensor may be arranged in the user control interface housing. The user control interface housing may be arranged between the first end and the second end of the tubing system. In one or more exemplary irrigation systems, the pump may be arranged in the user control interface housing. The second pressure sensor may be arranged in the user control interface housing.

A second tubing part of the tubing system may be for connecting the user control interface housing with a catheter and may comprise at least a part of the pressure channel.

The tubing system may comprise a second membrane. The second membrane may be arranged in the pressure channel. The first pressure sensor may be configured to measure the pressure in the pressure channel via the second membrane. The second pressure sensor may be configured to measure the pressure in the pressure channel via the second membrane.

The pressure channel may be a closed or sealed channel. The pressure channel may be filled with gas, such as air. The pressure in the pressure channel may be in the range from 1.0 to 4.0 Psi, e.g. at 25° C.

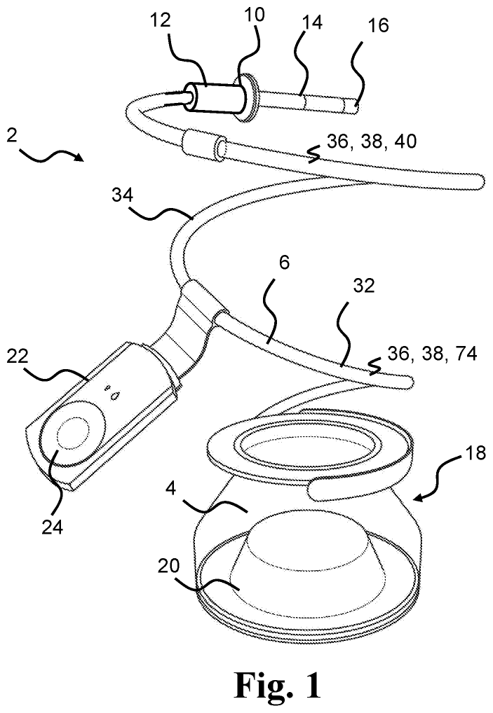

FIG. 1 shows an exemplary irrigation system for anal and/or stomal irrigation. The irrigation system 2 comprises a reservoir 4 for an irrigation liquid; a tubing system 6 with a first end 8 (not shown in FIG. 1) connectable to the reservoir 4, and a second end 10 with a second connector 12 and connectable to a catheter 14 for provision of a first fluid path for the irrigation liquid between the reservoir 4 and the catheter 14 comprising a distal end 16 for expelling irrigation liquid from the catheter 16. The irrigation system 2 comprises one or more pressure sensors including a first pressure sensor for provision of a first pressure signal. Further, the irrigation system 2 comprises a pump operable to pump the irrigation liquid from the reservoir 4 to the second end 10 of the tubing system 6. The pump is in irrigation system 2 arranged in reservoir housing 18 with reservoir 4, such as in bottom part 20 of the reservoir housing 18. The irrigation system 2 comprises a user interface housing 22 with a user interface 24 attached to the tubing system 6. The first pressure sensor is arranged in the user interface housing 22. The tubing system 6 comprises a first tubing part 32 and a second tubing part 34 each forming a part of the first fluid path of the irrigation system. The first tubing part 32 extends between the reservoir housing 18 and a user control interface housing 22 attached to the tubing system 6. The second tubing part 34 extends between the user control interface housing 22 and the second connector 12. The irrigation system 2 comprises a control system connected to the first pressure sensor and the pump. The control system is configured to operate the pump based on the first pressure signal from the first pressure sensor. The control system may be arranged in the user control interface housing 22 and/or in the reservoir housing 18.

The tubing system 6 comprises a pressure channel and a first membrane, wherein the first membrane is arranged in the second connector 12 of the tubing system between the first fluid path and the pressure channel. The pressure channel extends from the second connector 12 to the user interface housing 22 in the second tubing part 34, where the first pressure sensor is configured to measure the pressure in the pressure channel.

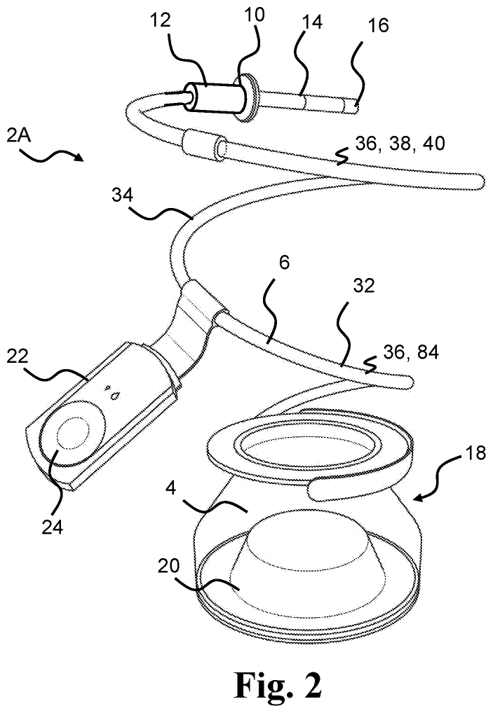

FIG. 2 shows an exemplary irrigation system for anal and/or stomal irrigation. The irrigation system 2A comprises a reservoir 4 for an irrigation liquid; a tubing system 6 with a first end 8 (not shown in FIG. 1) connectable to the reservoir 4, and a second end 10 with a second connector 12 and connectable to a catheter 14 for provision of a first fluid path for the irrigation liquid between the reservoir 4 and the catheter 14 comprising a distal end 16 for expelling irrigation liquid from the catheter 16. The irrigation system 2 comprises one or more pressure sensors including a first pressure sensor for provision of a first pressure signal. Further, the irrigation system 2 comprises a pump operable to pump the irrigation liquid from the reservoir 4 to the second end 10 of the tubing system 6. The irrigation system 2 comprises a user interface housing 22 with a user interface 24 attached to the tubing system 6. The pump and the first pressure sensor is in irrigation system 2A arranged in user interface housing 22. The tubing system 6 comprises a first tubing part 32 and a second tubing part 34 each forming a part of the first fluid path of the irrigation system. The first tubing part 32 extends between the reservoir housing 18 and a user control interface housing 22 attached to the tubing system 6. The second tubing part 34 extends between the user control interface housing 22 and the second connector 12. The irrigation system 2A comprises a control system connected to the first pressure sensor and the pump. The control system is configured to operate the pump based on the first pressure signal from the first pressure sensor. The control system may be arranged in the user control interface housing 22 and/or in the reservoir housing 18.

The tubing system 6 of irrigation system 2A comprises a pressure channel and a first membrane, wherein the first membrane is arranged in the second connector 12 of the tubing system between the first fluid path and the pressure channel. The pressure channel extends from the second connector 12 to the user interface housing 22 in the second tubing part 34, where the first pressure sensor is configured to measure the pressure in the pressure channel. The first tubing part 32 comprises a pump channel connected to the pump and the reservoir 4 for allowing the pump in the user control interface housing 22 to pump irrigation liquid from the reservoir 4 through the first fluid path to the catheter 14 connected to the second connector 12.

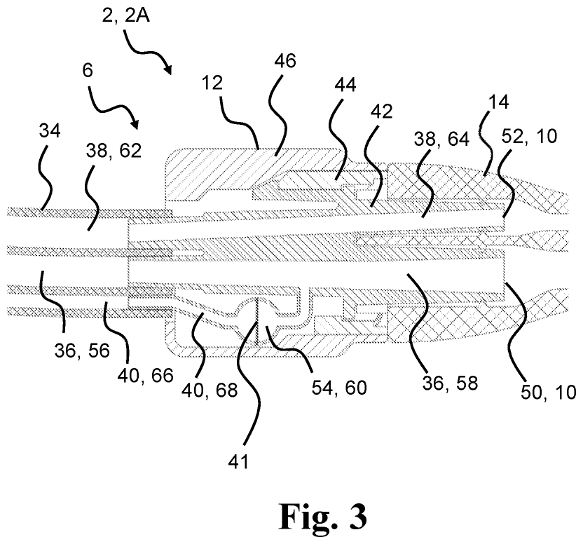

FIG. 3 illustrates a cross-sectional view of a part of an exemplary irrigation system. The irrigation system 2, 2A comprises a tubing system 6 with a first fluid path 36, a second fluid path 38, and a pressure channel 40. The first fluid path 36 is for feeding irrigation fluid from the reservoir to second end 10 of the tubing system, and the second fluid path 38 is for feeding second fluid to the second end 10 of the tubing system. The tubing system 6 comprises a second tubing part 34 and a second connector 12. The tubing system 6 comprises a first membrane 41 arranged between the first fluid path 36 and the pressure channel 40. The second connector comprises one or more connector parts including a first connector part 42, a second connector part 44 and a third connector part 46. The first connector part 42 is, in an assembled second connector 12, optionally snap-locked to and/or in threaded engagement with the second connector part 44. The second connector part 44 is, in an assembled second connector, optionally snap-locked to and/or in threaded engagement with the third connector part 46. The second connector 12 has a first outlet 50 of first fluid path 36 and a second outlet 52 of the second fluid path 38 (if present) of the tubing system 6. The first membrane 41 is arranged in an expansion chamber 54 of the first connector part 42. The first membrane 41 separates the first fluid channel 36 and the pressure channel 40. The first membrane is flexible to substantially equalize the pressure in the first fluid path and the pressure in the pressure channel, e.g. at least within a pressure difference less than a pressure difference threshold. The pressure difference threshold may be in the range from 0 to 0.5 psi. The pressure channel 40 may be a closed pressure channel, e.g. wherein the pressure in the closed pressure channel is in the range from 1.0 Psi to 4.0 Psi.

The second tubing part 34 comprises a first channel 56 and the first connector part 42 comprises a first channel 58, the channels 56, 58 forming a part of the first fluid path 36. The first channel 56 is in fluid communication with a first part 60 of the expansion chamber 54. The second tubing part 34 comprises a second channel 62 and the first connector part 42 comprises a second channel 64, the channels 62, 64 forming a part of the second fluid path 38. The second tubing part 34 comprises a pressure channel part 66 and the first connector part 42 comprises a pressure channel part 68 forming at least a part of the pressure channel 40.

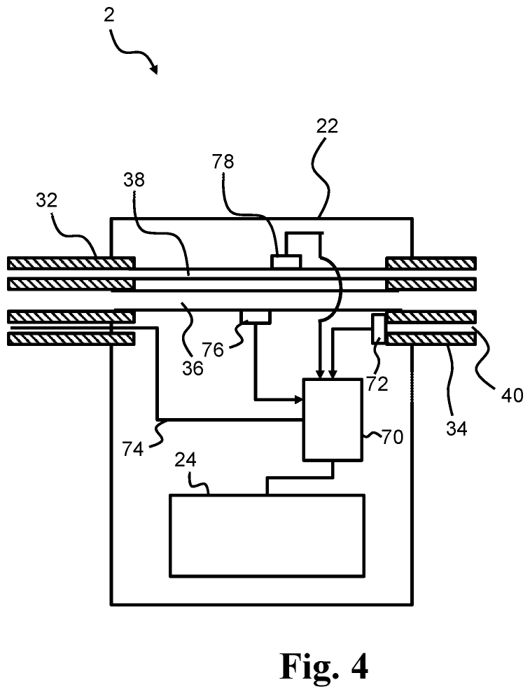

FIG. 4 schematically illustrates a part of an exemplary irrigation system. The irrigation system 2 comprises a tubing system including first tubing part 32 and second tubing part 34, the first and second tubing parts 32, 34 respectively connected to the user interface housing 22. A first fluid path 36 and a second fluid path 38 is provided in the irrigation system. The first fluid path 36 is used for feeding irrigation liquid from the reservoir to the catheter tip by connecting first outlet of the first fluid path to the catheter, see FIG. 3. The second fluid path 38 is used for feeding second fluid (for filling a catheter balloon) from the reservoir to the catheter balloon by connecting second outlet of the second fluid path to the catheter, see FIG. 3.

The irrigation system 2 comprises a control system including a controller 70, and a first pressure sensor 72 arranged in the user interface housing 22. The controller 70 is connected to the user interface 24 for receiving user input control signals and to the first pressure sensor 72 for receiving the first pressure signal indicative of the pressure in the pressure channel 40 from the first pressure sensor 72. Thus, the first pressure sensor 72 is configured to measure the pressure in the pressure channel 40. Arranging the first pressure sensor 72 in the user interface housing 22 is advantageous in order to provide a sufficiently accurate measurement of the pressure in the second connector 12. At the same time, the length of the pressure channel 40 is reduced, e.g. compared to a configuration with the first pressure sensor arranged in the reservoir housing. The controller 70 is connected to the pump arranged in the reservoir housing 18 by wired connection 74 and is configured to operate the pump (not shown) via one or more control signals on the wired connection 74. The wired connection 74 is optionally integrated in the first tubing part 32 as shown. The control system (controller 70) is configured to operate the pump (not shown) to reduce the irrigation liquid pressure in the first fluid path 36, if the pressure in the pressure channel 40 is larger than a first pressure threshold. The first pressure threshold may be 2.0 psi. In an exemplary irrigation system, the first pressure threshold is in the range from 1.6 to 2.2 psi.

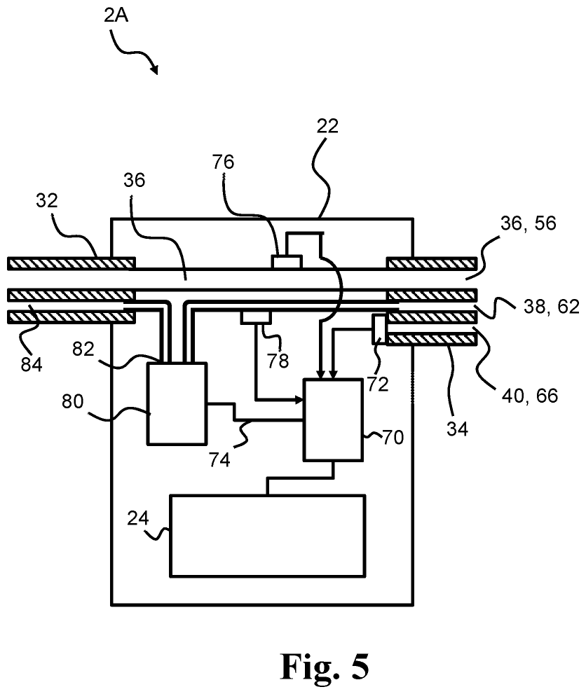

In FIGS. 4 and 5, the first pressure sensor 72 seals the pressure channel 40 at a first end of the second tubing part 34 to form a closed pressure channel filled with gas.

The control system (controller 70) is configured to operate the pump based on the first pressure signal indicative of the pressure in the pressure channel 40. Thus, the pump is operated in accordance with the pressure in the first liquid path 36 close to the catheter tip 16. Thereby, the necessary safety margin can be reduced, and the irrigation system can operate closer to the maximum irrigation liquid pressure at the catheter tip in accordance with regulatory requirements. Thus, an improved and more safe irrigation is provided.

Optionally, the irrigation system 2 comprises a second pressure sensor 76 arranged in the user interface housing 22 and configured to measure the pressure in the first fluid path 36 in the user interface housing 22. The second pressure sensor 76 is connected to the controller 70 for provision of a second pressure signal indicative of the pressure in the first fluid path of the user interface housing 22. The control system (controller 70) is optionally configured to operate the pump based on the second pressure signal indicative of the pressure in the first fluid path of the user interface housing 22. Thus, an improved and more safe irrigation is provided and/or detection of errors/malfunction is improved.

Optionally, the irrigation system 2 comprises a third pressure sensor 78 arranged in the user interface housing 22 and configured to measure the pressure in the second fluid path 38 in the user interface housing 22. The third pressure sensor 78 is connected to the controller 70 for provision of a third pressure signal indicative of the pressure in the second fluid path of the user interface housing 22. The control system (controller 70) is optionally configured to operate the pump based on the third pressure signal indicative of the pressure in the second fluid path of the user interface housing 22. Thus, an improved and more safe filling of the catheter balloon is provided.

The controller 70 may be configured to receive one or more reservoir pressure signals from one or more pressure sensors arranged in the reservoir housing 18 via wired connection 74. The control system (controller 70) is optionally configured to operate the pump based on the reservoir pressure signal(s). Thus, an improved and more safe irrigation is provided and/or detection of errors/malfunction is improved.

FIG. 5 schematically illustrates a part of an exemplary irrigation system where the pump 80 is arranged in the user interface housing 22. The irrigation system 2A comprises a tubing system including first tubing part 32 and second tubing part 34, the first and second tubing parts 32, 34 respectively connected to the user interface housing 22. A first fluid path 36 and a second fluid path 38 is provided in the irrigation system. The first fluid path 36 is used for feeding irrigation liquid from the reservoir to the catheter tip by connecting first outlet of the first fluid path to the catheter, see FIG. 3. The second fluid path 38 is used for feeding second fluid (for filling a catheter balloon) from the user interface housing 22 to the catheter balloon by connecting second outlet of the second fluid path to the catheter, see FIG. 3.

A first outlet 82 of the pump 80 is connected to a pumping channel 84 of the first tubing part 32. The pumping channel 84 is connected or connectable to the reservoir of the reservoir housing. Thereby, the pump 80 is configured to apply a reservoir pressure in the reservoir, the reservoir pressure in the reservoir thereby pressing irrigation liquid through the first fluid path 36 towards the catheter 14, see FIG. 2.

A second outlet 86 of the pump 80 is connected to the second fluid path 38 (second channel 62 of the second tubing part 34. Thereby, the pump 80 is configured to fill the catheter balloon with air as second fluid via the second fluid path 38.

The irrigation system 2A comprises a control system including a controller 70, and a first pressure sensor 72 arranged in the user interface housing 22. The controller 70 is connected to the user interface 24 for receiving user input control signals and to the first pressure sensor 72 for receiving the first pressure signal indicative of the pressure in the pressure channel 40 from the first pressure sensor 72. Thus, the first pressure sensor 72 is configured to measure the pressure in the pressure channel 40. Arranging the first pressure sensor 72 in the user interface housing 22 is advantageous in order to provide a sufficiently accurate measurement of the pressure in the second connector 12. At the same time, the length of the pressure channel 40 is reduced, e.g. compared to a configuration with the first pressure sensor arranged in the reservoir housing. The controller 70 is connected to the pump 80 arranged in the user interface housing 22 by wired connection 74 and is configured to operate the pump 80 via one or more control signals on the wired connection 74. The control system (controller 70) is configured to operate the pump 80 to reduce the irrigation liquid pressure in the first fluid path 36, if the pressure in the pressure channel 40 is larger than a first pressure threshold. The first pressure threshold may be 2.0 psi.

The control system (controller 70) is configured to operate the pump based on the first pressure signal indicative of the pressure in the pressure channel 40. Thus, the pump 80 is operated in accordance with the pressure in the first liquid path 36 close to the catheter tip 16. Thereby, the necessary safety margin can be reduced, and the irrigation system can operate closer to the maximum irrigation liquid pressure at the catheter tip in accordance with regulatory requirements. Thus, an improved and more safe irrigation is provided.

Optionally, the irrigation system 2 comprises a second pressure sensor 76 arranged in the user interface housing 22 and configured to measure the pressure in the first fluid path 36 in the user interface housing 22. The second pressure sensor 76 is connected to the controller 70 for provision of a second pressure signal indicative of the pressure in the first fluid path of the user interface housing 22. The control system (controller 70) is optionally configured to operate the pump 80 based on the second pressure signal indicative of the pressure in the first fluid path of the user interface housing 22. Thus, an improved and more safe irrigation is provided and/or detection of errors/malfunction is improved.

Optionally, the irrigation system 2 comprises a third pressure sensor 78 arranged in the user interface housing 22 and configured to measure the pressure in the second fluid path 38 in the user interface housing 22. The third pressure sensor 78 is connected to the controller 70 for provision of a third pressure signal indicative of the pressure in the second fluid path of the user interface housing 22. The control system (controller 70) is optionally configured to operate the pump based on the third pressure signal indicative of the pressure in the second fluid path of the user interface housing 22. Thus, an improved and more safe filling of the catheter balloon is provided.

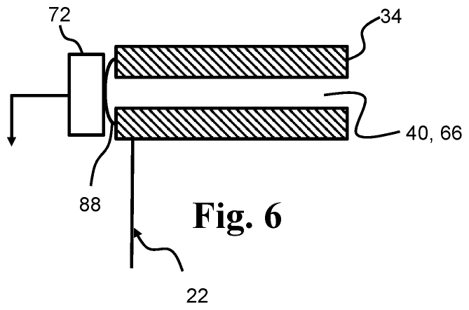

FIG. 6 shows a part of an exemplary irrigation system with a user interface housing 22, e.g. as shown in FIG. 4 or FIG. 5. The tubing system (second tubing part 34) is the same as in FIG. 4 or 5 with the additional feature, that the tubing system comprises a second membrane 88 arranged in the pressure channel 40. The second membrane 88 seals the pressure channel 40 at a first end of the second tubing part 34 to form a closed pressure channel filled with gas. The pressure in the pressure channel is in the range from 1.0 psi to 4.0 psi. The first pressure sensor 72 is configured to measure the pressure in the pressure channel 40 via the second membrane by the flexible second membrane 88 contacting and applying a pressure to the first pressure sensor 72.

Although particular features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications and equivalents.

### LIST OF REFERENCES

2 irrigation system
4 reservoir
6 tubing system
8 first end of tubing system
10 second end of tubing system
12 second connector
14 catheter
16 distal end of catheter
18 reservoir housing
20 bottom part
22 user interface housing
24 user interface
32 first tubing part
34 second tubing part
36 first fluid path
38 second fluid path
40 pressure channel
41 first membrane
42 first connector part
44 second connector part
46 third connector part
50 first outlet
52 second outlet
54 expansion chamber
56 first channel of second tubing part
58 first channel of first connector part
60 first part of expansion chamber
62 second channel of second tubing part
64 second channel of first connector part
66 pressure channel part of second tubing part
68 pressure channel part of first connector part
70 controller
72 first pressure sensor
74 wired connection
76 second pressure sensor
78 third pressure sensor
80 pump
82 first outlet of pump
84 pumping channel
86 second outlet of pump
88 second membrane

1. An irrigation system for anal or stomal irrigation, the irrigation system comprising:
a reservoir configured to contain a liquid;
a tubing system with a first end connectable to the reservoir, and a second end connectable to a catheter to form a first fluid path for the liquid to flow between the reservoir and the catheter, where the catheter comprises a distal end for expelling the liquid from the catheter;
a plurality of pressure sensors including a first pressure sensor adapted to output a first pressure signal;
a pump operable to pump the liquid from the reservoir to the second end of the tubing system; and
a control system connected to the first pressure sensor;
wherein the tubing system comprises a pressure channel and a first membrane, with the pressure channel separate from the first fluid path and the first membrane located between the first fluid path and the pressure channel;
wherein the control system is configured to operate the pump to control the flow of the liquid in the first fluid path for the anal or the stomal irrigation based on a sensed pressure in the pressure channel as sensed by the first pressure signal.

2. The irrigation system according to claim 1, wherein the tubing system comprises a second connector at the second end, and the first membrane is arranged in the second connector.

3. The irrigation system according to any of claims 1-2, wherein the irrigation system comprises a user control interface housing attached to the tubing system, wherein the first pressure sensor is disposed inside of the user control interface housing.

4. The irrigation system according to claim 1, wherein the first pressure sensor is disposed inside of a reservoir housing accommodating the reservoir.

5. The irrigation system according to claim 5, wherein the tubing system comprises a second fluid path parallel to the first fluid path, with the second fluid path communicating with an inflatable balloon of the catheter.

6. The irrigation system according to any claim 5, further comprising a second pressure sensor, wherein the control system is connected to the second pressure sensor and is configured to operate the pump for inflation of the inflatable balloon based on a signal from the second pressure sensor.

7. The irrigation system according to claim 6, wherein the second pressure sensor is disposed inside of the user control interface housing.

8. The irrigation system according to claim 1, wherein the first pressure signal indicates a relative pressure between the sensed pressure in the pressure channel and an ambient pressure.

9. The irrigation system according to claim 1, wherein the first pressure signal indicates an absolute pressure in the pressure channel.

10. The irrigation system according to claim 1, wherein the tubing system comprises a second membrane arranged in the pressure channel, and wherein the first pressure sensor is configured to measure a pressure in the pressure channel as sensed by the second membrane.

11. The irrigation system according to claim 1, wherein the pressure channel is filled with gas.

12. (canceled)

13. The irrigation system according to claim 1, wherein the control system is configured to operate the pump to reduce an irrigation liquid pressure in the first fluid path if the sensed pressure in the pressure channel is larger than a pre-determined pressure threshold.

14. (canceled)

15. An irrigation system for anal or stomal irrigation, the irrigation system comprising:
a reservoir configured to contain a liquid;
a tubing system providing a first flow path between the reservoir and a catheter, and a pressure channel separate from the first flow path;
a pump operable to move the liquid through the first flow path from the reservoir and through the catheter;
a control system connected to the tubing system;
a connector coupling the tubing system to the catheter, where the connector includes a chamber coupling the pressure channel to the first flow path, with a membrane located in the chamber between the first flow path and the pressure channel; and
a pressure sensor communicating with the control system, with the pressure sensor configured to sense a pressure in the first flow path;
wherein the control system operates the pump to control the anal or the stomal irrigation with the liquid based on the pressure sensed in the first flow path.
